(19) United States
(12) Patent Application Publication
(10) Pub. No.: US 2007/0112324 A1
(43) Pub. Date: May 17, 2007

(54) Cap for safety packaging device for a bottle for medical use

(75) Inventor: Farid Hamedi-Sangsari, Lyon (FR)

Correspondence Address: NIXON & VANDERHYE, PC, 901 NORTH GLEBE ROAD, 11TH FLOOR, ARLINGTON, VA, 22203, US

(73) Assignee: M.A.P. FRANCE, Lyon (FR)

(21) Appl. No.: 11/596,369

(22) PCT Filed: Dec. 3, 2004
(86) PCT No.: PCT/FR04/03117

§ 371 Date: Nov. 14, 2006

(30) Foreign Application Priority Data
Dec. 5, 2003 (FR) 0314295

(51) Int. Cl. A61M 5/32

(52) U.S. Cl. 604/414

(57) ABSTRACT

The invention relates to a perforable cap pre-mounted on the end (D) of the double-tipped needle of a safety packaging device for a bottle for medical use. This cap is slidingly mounted in a sealed manner so as to prevent an aerosol effect when the stopper of the bottle or, for example, the injection site of a perfusion bag is perforated.

The present invention relates to a perforable cap pre-mounted on the needle of a safety packaging device for a bottle for medical use. This cap is slidingly mounted in a sealed manner so as to prevent an “aerosol” effect when the stopper of the bottle or, for example, the injection site of a perfusion bag is perforated.

The present invention is an improvement to patent application FR2828803 by the same applicant. This application describes a safety packaging device for a bottle for medical use. It consists of a cylindrical one-piece plastic body with two parts: a lower part designed to cover the glass bottle completely and optionally comprising at its base means for stabilizing the device in the vertical position, and an upper part of smaller diameter consisting of a cylindrical chamber comprising means, for example a double-tipped needle, for transferring the contents of the bottle into a container, such as a perfusion bag. The aim of the device described in this application is to offer optimum safety as regards the risk of the bottle breaking, the risk of accidental sticking with the needle and the risk of leakage.

The substances contained in this type of bottle are potentially dangerous to users or the environment (antineoplastics, antibiotics, etc.). It is therefore very important to prevent contact with these substances.

When bottles for medical use which contain a powdered active substance are used, an “aerosol” effect may occur owing to the fact that the bottle with the powdered drug contains a certain amount of air which is trapped when the stopper is sealed on.

The dispensing/stoppering operation is carried out in a clean room at a pressure slightly above the atmospheric pressure of the geographical location and at a controlled ambient temperature of around 20° C.

When the device is used at a geographical location having an atmospheric pressure which is lower than that at which the bottle was stoppered (difference in altitude for example), the pressure inside the bottle becomes positive in relation to the ambient air, causing powder to spray outward like an aerosol when the stopper of the bottle is perforated. Overpressures in the bottle of up to 100 mbar have been measured. The same effect occurs when the air contained in the bottle is warmer than originally (law of expansion of gases).

If these powders, which are often allergenic, or even toxic (antibiotics, antineoplastics), are sprayed near care staff and the patient, it can be very harmful to their health and must therefore be prevented.

There is no existing simple technical solution to solve this problem. A number of patent documents describe systems for protecting the end of a needle or facilitating the transfer of the contents of a syringe.

Patent GB2359754 describes a sheath which covers the needle of a hypodermic syringe in a sealed manner and which can perforate the rubber stopper of a vial.

Publication WO0126718 describes a device for reconstituting a preparation using a hollow transfer element such as a needle. The device eliminates any risk of nebulization or accidental spraying of the product when it is connected to the injection needle.

However, no patent describes a system similar to the one of the invention for preventing the “aerosol” effect that occurs when a container containing a powdered product is placed in communication with another container, for example a perfusion bag.

The present invention makes it possible to solve the problem mentioned above by adding, to an existing transfer device, a simple part that does not entail a significant extra cost in relation to the whole.

This part is a perforable cap which is slidingly pre-mounted in a sealed manner on one end of the double-tipped needle of a safety packaging device for a bottle for medical use. The fit is designed in such a way as to ensure that the cap is held in place, that the emerging end of the needle is sealed off from the surrounding environment, and that the cap can slide on the needle body. The friction/sealing functions can be performed for example by an annular bulge.

The attached drawings will illustrate the present invention in greater detail.

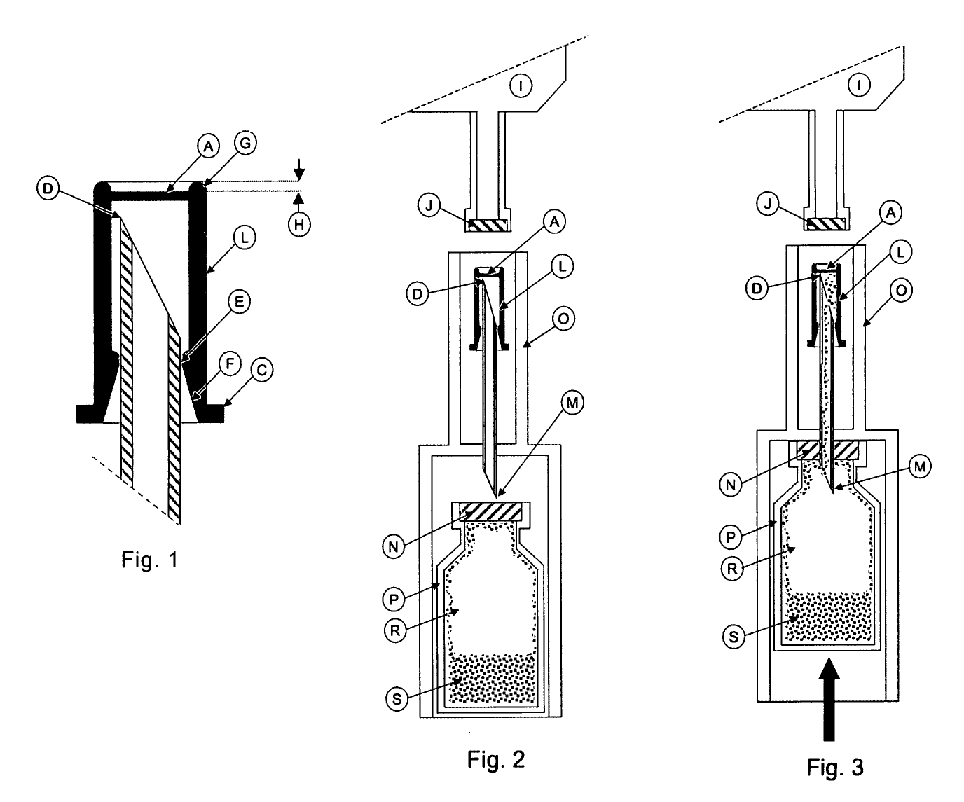

FIG. 1 is a view in longitudinal section of the cap of the invention, mounted on one of the ends (D) of a double-tipped needle.

FIG. 2 is a view in longitudinal section of the safety packaging device (O) equipped with the cap of the invention (L) with a partial sectional view of the perfusion bag (I) with its injection site (J), before use.

FIG. 3 is a partial sectional view of the system after the tip (M) of the double-tipped needle has pierced the stopper (N) of the bottle (P).

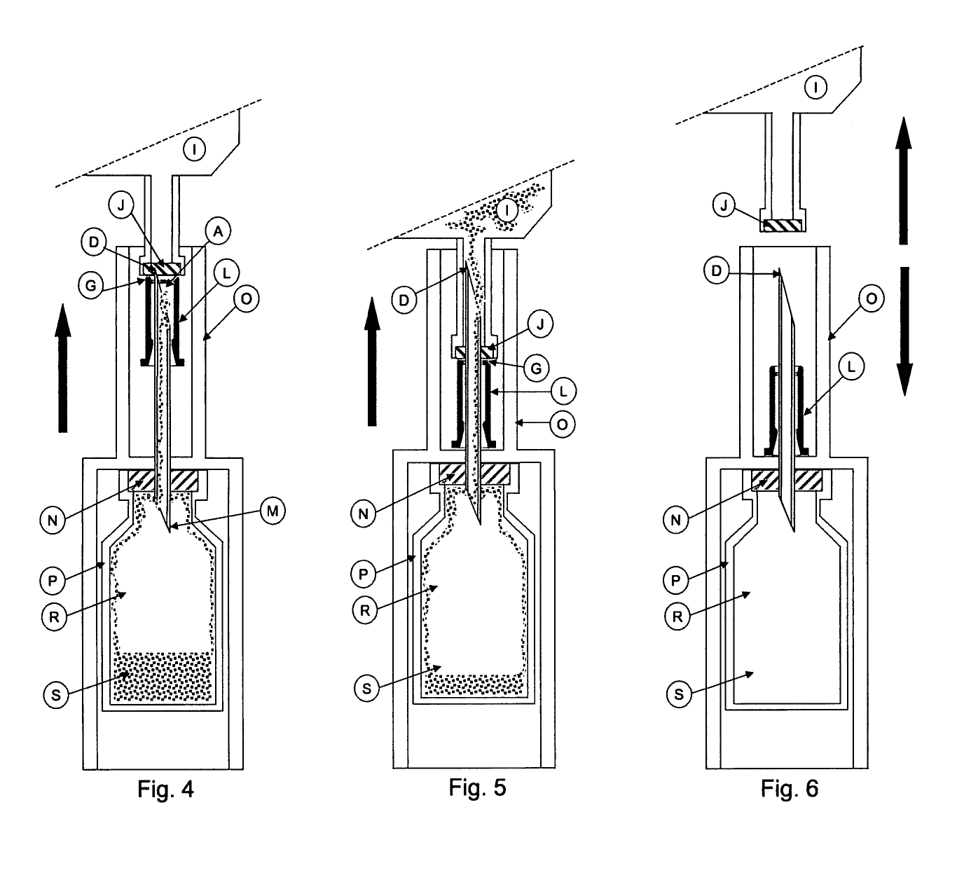

FIG. 4 is a partial sectional view of the system at the start of perforation of the end wall of the cap (A) and the injection site (J) by the tip (D) of the double-tipped needle.

FIG. 5 is a partial sectional view of the system after the site (J) has been completely perforated and the cap (L) has slid over a length sufficient to place the contents of the bag (I) and of the bottle (P) in communication.

FIG. 6 is a partial sectional view of the system after the device (O) and the bag (I) have been disconnected after use.

FIG. 1 shows the cap (L) according to the present invention mounted on the end (D) of a needle. The cap (L) of generally cylindrical shape comprises an end wall (A) and an open end for mounting it on a needle. The end wall (A) of the cap (L) is thin enough to be easily perforated by the tip (D) of the needle. Preferably, the end wall (A) of the cap (L) is set back (H) from the end of the cap (L), so as to create one or more sealing ring(s) (G) for improving the seal at the point of contact between the cap and the injection site of the perfusion bag. The open end of the cap comprises a centering cone (F) designed to facilitate its fitting on the tip of the needle and a collar (C) to facilitate the dispensing of the part for the purposes of automatic assembly. Preferably, the cap comprises sealing means allowing the cap to slide on the body of the needle. These sealing means may consist of one or more annular bulges (E) located above the centering cone (F).

FIG. 2 shows the system used in the invention as a whole: safety packaging device (O) with the cap (L) and perfusion bag (I) with injection site (J). The safety packaging device (O) comprises a double-tipped needle with two ends (D, M), and it completely covers the bottle (P) which is closed by a perforable stopper (N) and contains a powdered active substance (S). The amount of powder (S) is such that there is a volume of free air (R) in the bottle (P).

FIG. 3 shows a view of the system after the stopper (N) of the bottle (P) has been pierced by the lower tip (M) of the double-tipped needle. The difference between the conditions prevailing inside the bottle and in the external environment (pressure and/or temperature) causes some of the powder (S) to spray through the needle into the cap (L). This powder remains trapped in a sealed manner inside the cap.

FIG. 4 shows a view of the system as the tip (D) of the double-tipped needle starts to perforate the end wall of the cap (A) and the injection site (J). When the injection site (J) of the perfusion bag (I) is inserted into the safety packaging device (O), the end (D) of the double-tipped needle pierces first of all the end wall of the cap (A), then the injection site (J). The sealing ring (G) of the cap (L) bears on the elastomer disc of the injection site (J). Sealing in the perforated region is guaranteed by the bearing force, needed to perforate both the end wall of the cap and the injection site, applied to the sealing ring (G) of the cap against the elastomer seal of the injection site (J). There will therefore be no outward “aerosol” effect.

FIG. 5 shows a view of the system after the site (J) has been completely perforated and the cap (L) has slid. The cap (L) slides along the needle body until the end of the double-tipped needle has completely perforated the injection site, for example until the collar (C) of the cap (L) comes into abutment against the partition that bears the double-tipped needle. The end (D) of the needle passes right through the injection site (J) so as to place the contents of the bag (I) and of the bottle (P) in communication. Powder (S) is sprayed (“aerosol” effect) inside the bag and does therefore not contaminate the external environment.

FIG. 6 shows a view of the system after use. Since the elastomer seal of the injection site (J) is self-sealing, the contents of the bag (I) are in a closed medium after the needle is removed.

The cap (L) of the present invention is preferably made of a flexible or semi-rigid plastic (polyethylene, polypropylene, elastomer, etc.).

In the embodiment shown in the figures, the cap is mounted on the end (D) of the double-tipped needle that will be placed in communication with the perfusion bag (J). As a variant, the cap may be mounted on the other end (M) of the needle. In this case, it is necessary to first of all perforate the injection site (J) of the perfusion bag (I) directly, then simultaneously perforate the end wall (A) of the cap (L) and the stopper (N) of the bottle (P). In this embodiment, it is necessary for the lower part of the double-tipped needle, i.e. the portion of the needle that penetrates the free volume (R) of the bottle (P), to be sufficiently long to allow the cap (L) to be positioned and to slide along the needle body. In another variant, a cap (L) could be provided on both ends (D, M) of the needle.

The description and the figures illustrate different embodiments of the present invention. The invention is not however limited to the embodiments described and shown but, on the contrary, encompasses all variants.

1. A device which is mounted on at least one of the ends (D, M) of a double-tipped needle of a safety packaging (O) for a bottle for medical use, for directly transferring the contents of a bottle (P) into a container, such as a perfusion bag (I), characterized in that it consists of an easily perforable cap (L) which is slidingly pre-mounted in a sealed manner on at least one of the ends (D, M) of the double-tipped needle, the end wall (A) of the cap being perforated by the bearing force applied either to the seal of the injection site (J) or to the stopper (N) of the bottle (P), the cap (L) sliding on the needle body.

2. The device as claimed in claim 1, characterized in that the cap (L) comprises sealing means allowing the cap to slide on the body of the needle in a sealed manner.

3. The device as claimed in claim 2, characterized in that the sealing means consist of one or more annular bulges (E).

4. The device as claimed in claim 1, characterized in that the perforable end wall (A) of the cap (L) is set back (H) from the end of the cap, so as to create a sealing ring (G) for improving the seal at the point of contact between the cap and the injection site (J) of the perfusion bag (I) and/or at the point of contact between the cap and the stopper (N) of the bottle (P).

5. The device as claimed in claim 1, characterized in that the open end of the cap (L) comprises a centering cone (F) designed to facilitate its fitting on the tip of the needle.

6. The device as claimed in claim 1, characterized in that the open end of the cap (L) comprises a collar (C).

7. The device as claimed in claim 1, characterized in that the cap (L) is made of a flexible or semi-rigid plastic or elastomer.
